(19) United States
(12) Patent Application Publication
(10) Pub. No.: US 2011/0082074 A1
(43) Pub. Date: Apr. 7, 2011

(54) AGENT FOR PREVENTING PERIODONTAL DISEASE

(75) Inventors: Kimio NISHIMURA, Kyotanabe-shi (JP); Junji MORITA, Kyotanabe-shi (JP); Mayumi HIROSE, Kanazawa-shi (JP); Toshiyuki MURAI, Osaka (JP)

(73) Assignee: THE DOSHISHA, Kyoto-shi (JP)

(21) Appl. No.: 12/785,942

(22) Filed: May 24, 2010

(30) Foreign Application Priority Data
Oct. 7, 2009 (JP) 2009-233379

(51) Int. Cl. A61K 38/16; C07K 14/42; A61P 31/04

(52) U.S. Cl. 514/2.8; 530/370; 530/378; 530/371

(57) ABSTRACT

The present invention provides a pharmaceutical agent that is useful for preventing periodontal disease. The pharmaceutical agent contains a compound in which a mucin-type sugar chain-binding lectin is bound to a polypeptide having an integrin recognition sequence.

### TECHNICAL FIELD

The present invention mainly relates to a pharmaceutical agent that is useful for preventing periodontal disease; an oral composition comprising the pharmaceutical agent; and a tooth-cleaning apparatus comprising the pharmaceutical agent.

### BACKGROUND ART

Periodontal disease is a chronic inflammatory lesion caused by periodontal pathogens that inhabit dental plaque, which is a biofilm formed on teeth (see Non-Patent Literatures (NPL)1 and 2). The periodontal disease-causing bacteria Porphyromonas gingivalis (hereinafter referred to as “P. gingivalis”) is believed to have the most potent pathogenicity. The fimbriae of Porphyromonas gingivalis are considered to be involved in the exhibition of various pathogenicities. Prof. Atsuo Amano of Osaka University, Graduate School of Dentistry, classified fimA genes encoding fimbrial subunit FimA into six types of polymorphisms (types I, Ib, II, III, IV, and V), based on the variations in the nucleotide sequences thereof. Prof. Amano has revealed clinical correlations between periodontitis and P. gingivalis carrying type II fimA gene in adult patients with periodontal disease (see Non-Patent Literature (NPL) 3), intellectually disabled persons with periodontal disease (see Non-Patent Literature (NPL) 4), Down's syndrome patients with periodontal disease (see Non-Patent Document 5), and type 2 diabetes patients with periodontal disease (see Non-Patent Literature (NPL) 4). More specifically, Prof. Amano revealed that periodontal disease patients carry P. gingivalis predominantly carrying type II fimA, and develop periodontal disease, resulting in the progression of periodontitis.

Prof. Amano further revealed that the type II FimA fimbrial protein of P. gingivalis carrying type II fimA gene adheres to epithelial cells via α5β1 integrin of the epithelial cells, and invades the epithelial cells to cause cell damage (see Non-Patent Literature (NPL) 6).

The removal of P. gingivalis, once spread in the oral cavity, is currently believed to be difficult. Particularly when a person is infected with P. gingivalis carrying type II fimA gene, the infected person is considered to most likely develop periodontal disease in the future. Although the rate of infected persons is extremely high, it has been difficult to retain a pharmaceutical agent for P. gingivalis in the oral cavity for a long period of time; therefore, considerable care and plaque control have been required to inhibit the development and progression of periodontal disease.

### CITATION LIST

### Non-Patent Literatures

[NPL 1] Socransky S S, and Haffajee A D, (1994): Evidence of bacterial etiology: a historical perspective. Periodontol 2000 5, 7-25
[NPL 2] Socransky S S, and Haffajee A D, (2005): Periodontal microbial ecology. Periodontol 2000 38, 135-87
[NPL 3] Tamura K, Nakano K, Nomura R, Miyake S, Nakagawa I, Amano and Ooshima T. (2005): Distribution of Porphyromonas gingivalis fimA genotypes in Japanese children and adolescents. Journal of Periodontology 76, 674-679
[NPL 4] Amamo A, Nakagawa I, Okahashi N and Hamada N (2004): Variations of Porphyromonas gingivalis fimbriae in relation to microbial pathogenesis. Journal of Periodontal Research 39, 136-142
[NPL 5] Ojima M, Takeda M, Yoshioka H, Nomura M, Tanaka N, Kato T, Shizukushi, S and Amano, A (2005): Relationship of periodontal bacterium genotypic variation with periodontitis in type 2 diabetic patients. Diabetes Care 28, 433-434
[NPL 6] Hiroaki Inaba, Munehiro Takeda, Atsuo Amano, Journal of the Japanese Society of Periodontology 47: 164, 2005 “Functional differences among FimA variants of Porphyromonas gingivalis and their effects on cytotoxicity to and invasion of human epithelial cells”.

### SUMMARY OF INVENTION

### Technical Problem

A main object of the present invention is to provide a pharmaceutical agent that is useful for preventing periodontal disease.

### Solution to Problem

The present inventors focused on the fact that the acquired pellicle in the oral cavity mainly consists of a mucin-type glycoprotein, and found that when a lectin that binds to a mucin-type sugar chain is bound to a component that inhibits binding of periodontal disease-causing bacteria to integrin, the binding inhibitory component can be retained on the acquired pellicle, which is a periodontal disease lesion, so that a pharmaceutical agent that is effective for preventing periodontal disease can be produced. The present inventors conducted further research, and accomplished the present invention.

More specifically, the present invention provides the inventions itemized below:

Item 1. A compound comprising a mucin-type sugar chain-binding lectin and a polypeptide bound thereto, the polypeptide comprising an integrin recognition sequence.
Item 2. The compound according to Item 1, wherein the mucin-type sugar chain-binding lectin is at least one lectin selected from the group consisting of mushroom lectin, Dolichos biflorus lectin, peanut lectin, and soybean lectin.
Item 3. The compound according to Item 1 or 2, wherein the polypeptide comprising the integrin recognition sequence comprises an amino acid sequence set forth in SEQ ID NO: 1 or 2.
Item 4. The compound according to Item 3, wherein the polypeptide comprising the amino acid sequence set forth in SEQ ID NO: 1 is at least one member selected from the group consisting of the polypeptide consisting of the amino acid sequence set forth in SEQ ID NO: 3, fibronectin, vitronectin, and laminin.
Item 5. The compound according to any one of Items 1 to 4 comprising, as an active ingredient, a compound in which a mucin-type sugar chain-binding lectin and a polypeptide comprising an integrin recognition sequence are bound via beads.
Item 6. The compound according to any one of Items 1 to 5 comprising, as an active ingredient, a compound in which a mucin-type sugar chain-binding lectin and a polypeptide comprising an integrin recognition sequence are bound at a lectin:polypeptide molar ratio of 1:0.01 to 1:10.
Item 7. A composition for the oral cavity comprising the compound of any one of Items 1 to 6.
Item 8. A tooth-cleaning apparatus comprising the composition for the oral cavity of Item 7.
Item 9. A method for inhibiting binding of Porphyromonas gingivalis to gingival cells, comprising applying an effective amount of the compound of any one of Items 1 to 6 to the oral cavity of a human or animal subject.
The method of Item 9 includes the methods described below in Items 9-1 to 9-8.
Item 9-1. A method for inhibiting binding of Porphyromonas gingivalis to gingival cells, comprising applying to the oral cavity of a human or animal subject a pharmaceutically effective amount of a compound comprising a mucin-type sugar chain-binding lectin and a polypeptide bound thereto, the polypeptide comprising an integrin recognition sequence.
Item 9-2. The method according to Item 9-1, wherein the mucin-type sugar chain-binding lectin is at least one type of lectin selected from the group consisting of mushroom lectin, Dolichos biflorus lectin, peanut lectin, and soybean lectin.
Item 9-3. The method according to Item 9-1 or 9-2, wherein the polypeptide comprising the integrin recognition sequence comprises an amino acid sequence set forth in SEQ ID NO: 1 or 2.
Item 9-4. The method according to Item 9-3, wherein the polypeptide comprising the amino acid sequence set forth in SEQ ID NO: 1 is at least one member selected from the group consisting of the polypeptide consisting of the amino acid sequence set forth in SEQ ID NO: 3, fibronectin, vitronectin, and laminin.
Item 9-5. The method according to any one of Items 9-1 to 9-4, wherein the compound comprising the mucin-type sugar chain-binding lectin and the polypeptide comprising an integrin recognition sequence is a compound in which the lectin and the polypeptide are bound via beads.
Item 9-6. The method according to any one of Items 9-1 to 9-5, wherein the compound comprising the mucin-type sugar chain-binding lectin and the polypeptide comprising an integrin recognition sequence is a compound in which the lectin and the polypeptide are bound at a lectin:polypeptide molar ratio of 1:0.01 to 1:10.
Item 9-7. The method according to any one of Items 9-1 to 9-6, wherein the human or animal subject is a periodontal disease patient or a periodontal disease mammal.
Item 9-8. The method according to any one of Items 9-1 to 9-7, wherein the effective amount is such that the mucin-type sugar chain-binding lectin contained in the compound is administered in an amount of 0.1 to 10 mmol per day.

The present invention is described below in more detail.

In this specification, “prophylactic . . . for periodontal disease” or “preventing periodontal disease” means both the prevention of the development of periodontal disease (development prevention), and the prevention of an already-developed periodontal disease condition or lesion from worsening (progression prevention).

1. Prophylactic Agent for Periodontal Disease

The prophylactic agent for periodontal disease contains, as an active ingredient, a compound comprising a mucin-type sugar chain-binding lectin and an integrin recognition sequence.

The mucin-type sugar chain-binding site refers to a site bound to a mucin-type sugar chain.

In particular, the “mucin-type sugar chain-binding site” in the compound used as an active ingredient of the prophylactic agent for periodontal disease of the present invention refers to a site derived from a mucin-type sugar chain-binding lectin. The mucin-type sugar chain-binding site interacts with the acquired pellicle mainly consisting of a mucin-type glycoprotein and formed in the oral cavity, thereby retaining the compound on the acquired pellicle for a prolonged period of time. Thus, the mucin-type sugar chain-binding site can also function to retain the integrin recognition site of the compound on the acquired pellicle for a prolonged period of time.

Further, the mucin-type sugar chain-binding site can inhibit the binding of P. gingivalis carrying type II FimA fimbrial protein to gingival cells. Therefore, the mucin-type sugar chain-binding site can also function to enhance the inhibitory effect of the integrin recognition site on binding of P. gingivalis to gingival cells.

Mucin is a type of glycoprotein produced in cells. Mucin is a major glycoprotein with which lumens of the oral cavity, trachea, digestive tracts such as stomach and intestines, etc. are covered.

The sugar chain contained in glycoprotein is classified into O-type and N-type sugar chains according to the protein binding form. Among these, most of the sugar chains present in mucins are O-type sugar chains that are formed by linking a sugar chain via an O-glycoside bond to the hydroxyl group on a serine or threonine residue of a peptide or protein. Therefore, an “O-type sugar chain” is also called a “mucin-type sugar chain”. That is, a “mucin-type sugar chain” is another name for an “O-type sugar chain”. In contrast, N-type sugar chains have a structure in which a sugar chain is bound to an asparagine residue of a protein or a peptide.

The “mucin-type sugar chain-binding lectin” as used herein refers to a lectin that can bind to a mucin-type sugar chain, i.e., an O-type sugar chain.

The mucin-type sugar chain may have various core structures. For example, a core 1 structure is formed by linking galactose to the hydroxyl group at the C3 of N-acetylgalactosamine via β1-3 linkage. A core 3 structure is formed by linking N-acetylglucosamine to the hydroxyl group at the C3 of the N-acetylgalactosamine via β1-3 linkage. A core 5 structure is formed by linking N-acetylgalactosamine to N-acetylgalactosamine via β1-3 linkage. A core 6 structure is formed by linking N-acetylglucosamine to N-acetylgalactosamine via β1-6 linkage. Various core structures can be produced by these combinations. The stem region is composed of the disaccharides Gal(β1-3)GlcNAc (type 1) and Gal(β1-4)GlcNAc (type 2). The type 1 and 2 sugar chains are branched by the linking of N-acetylglucosamine to galactose via β1-6 and β1-3 linkages. The sugar chain of the stem region terminates with a peripheral sugar, typically α-linked galactose, N-acetylgalactosamine, fucose or sialic acid.

Examples of glycoproteins that have a mucin-type sugar chain in the oral cavity include Mucin1, Mucin5B, and Mucin7.

The mucin-type sugar chain-binding lectin can specifically recognize a mucin-type sugar chain, and binds thereto.

The lectin includes various lectins derived from animals, plants, fungi, etc. The mucin-type sugar chain-binding lectin that is used in the present invention is not particularly limited in structure or origin, insofar as the lectin can specifically recognize a mucin-type sugar chain. Soybean lectin (SBA) is particularly preferable as the mucin-type binding lectinin of the present invention, because soybean lectin can reliably recognize a wide range of mucin-type sugar chains having an N acetylgalactosamine α1→3 galactosamine (GalNAc alpha1-3Gal) structure.

Examples of mucin-type binding lectins include, but are not limited to, mushroom lectin: ABA (Agaricus bisporus agglutinin); Dolichos biflorus lectin: DBA (Dolichos biflorus agglutinin); peanut lectin: PNA (Arachis hypogaea agglutinin); and soybean lectin: SBA (soybean agglutinin). These lectins are commercially available, and can be purchased, for example, from J-Oil Mills, Inc., Seikagaku Corporation, etc.

In the present invention, the “integrin recognition site” refers to a site derived from the integrin recognition sequence of a polypeptide comprising an integrin recognition sequence. The integrin recognition site may consist of only an integrin recognition sequence, or may additionally comprise a structure other than the integrin recognition sequence (for example, an amino acid sequence other than the integrin recognition sequence), insofar as the integrin recognition site can recognize integrin. The integrin recognition site inhibits the binding of a fimbriae subunit type II FimA fimbriae protein to integrin, thus being effective for plaque control.

Furthermore, the compound comprising a mucin-type sugar chain-binding site and an integrin recognition site can inhibit the production of TGF-β as well as the activation of a TNF receptor, thus inhibiting the development of gingival inflammation.

The peptide comprising an integrin recognition sequence comprises a specific amino acid sequence that recognizes integrin. In the compound used as an active ingredient of the prophylactic agent for periodontal disease of the present invention, the polypeptide comprising an integrin recognition sequence functions to recognize integrin.

Examples of the integrin recognition sequence include an amino acid sequence represented by arginine-glycine-aspartic acid (SEQ ID NO: 1 in the Sequence Listing) (hereinafter referred to as the “RGD sequence”), and an amino acid sequence represented by arginine-histidine-serine-arginine-asparagine (SEQ ID NO: 2 in the Sequence Listing) (hereinafter referred to as the “RHSRN sequence”). More specifically, examples of the polypeptide having an integrin recognition sequence include peptides comprising the RGD sequence, and peptides comprising the RHSRN sequence.

Examples of the integrin recognized by the polypeptide comprising an integrin recognition sequence include α5β1 integrin.

Examples of the polypeptide comprising an integrin recognition sequence include proteins.

In the compound used as an active ingredient of the prophylactic agent for periodontal disease of the present invention, the polypeptide comprising an integrin recognition sequence may consist of only an integrin recognition sequence (for example, the RGD sequence or the RHSRN sequence), or may comprise an integrin recognition sequence and other amino acid residues bonded thereto. In such a peptide, the other amino acid residues may be bound to either one or both of the C-terminus and N-terminus. The peptide may be a cyclic peptide. Examples of such amino acid residues include glycine (G), alanine (A), valine (V), proline (P), leucine (L), isoleucine (I), phenylalanine (F), tryptophan (W), methionine (M), serine (S), threonine (T), asparagine (N), glutamine (Q), tyrosine (Y), cysteine (C), aspartic acid (D), glutamic acid (E), ricin (K), arginine (R), histidine (H), and the like. In the peptide, the side chain of an amino acid residue other than the integrin recognition sequence may be protected or substituted by an appropriate functional group. Moreover, the polypeptide may contain one or two or more integrin recognition sequences, or may contain two or more identical integrin recognition sequences.

The polypeptide comprising an integrin recognition sequence is preferably bound to a mucin-type sugar chain-binding lectin in such a manner that the binding site (amino acid residue) is not within the integrin recognition sequence region. The integrin recognition sequence is preferably located far away from the binding site (i.e., in a region that is at least several to several tens of amino acid residues away from the binding site). Particularly, when an terminal amino acid of the polypeptide comprising an integrin recognition sequence (an amino acid within the region of up to several amino acid residues, preferably 1 to 3 residues, from the terminal end) is used for the binding, the integrin recognition sequence is preferably located at least several to several tens of amino acid residues away from the terminal amino acid. The integrin recognition sequence is particularly preferably located on a terminal side opposite the terminal side where the terminal amino acid used for binding is present. For example, when a C-terminal amino acid of the polypeptide having an integrin recognition sequence is used for the binding, the integrin recognition sequence is preferably present on the N-terminal side.

One particularly preferable example of the polypeptide comprising an integrin recognition sequence is a peptide consisting of the amino acid sequence of SEQ ID NO: 3 (RGDSPASSKP). This peptide comprises an RGD sequence consisting of three amino acid residues at the N-terminus. For example, when the peptide consisting of the amino acid sequence of SEQ ID NO: 3 is used, the peptide comprises an RGD sequence at the N-terminus. Therefore, an amino acid residue that is present on the C-terminal side (for example, proline (P) or lysine (K)) is preferably used for the binding to a mucin-type sugar chain-binding lectin.

It is also possible to use, as the polypeptide comprising an integrin recognition sequence, integrin-recognizing peptides described in the following two publications:

Erkki Koivunen et al., The Journal of Biological Chemistry, vol. 268, No. 27, pp. 20205-20210, 1993; and Erkki Koivunen et al., The Journal of Cell Biology, volume 124, No. 3, 373-380, 1994.

When the polypeptide comprising an integrin recognition sequence is a protein, the protein may be a natural or synthetic protein. Alternatively, the protein may be a conjugated protein comprising a sugar chain and a fatty acid bonded thereto. Examples of proteins comprising an RGD sequence as an integrin recognition sequence include fibronectin, vitronectin, laminin, and the like.

The length of the polypeptide comprising an integrin recognition sequence is not particularly limited, insofar as the polypeptide can recognize integrin. The polypeptide is typically 3 to 3,000 amino acid residues in length. When α represents an integer of from 3 to 3,000, the polypeptide is preferably α amino acid residues in length. The peptide is more preferably 2,500 residues or less in length. However, the peptide is also preferably 5 residues or more in length, more preferably 10 residues or more in length, and even more preferably 20 residues or more in length.

The compound used as an active ingredient of the prophylactic agent for periodontal disease of the present invention is formed by binding a mucin-binding lectin and a polypeptide comprising an integrin recognition sequence, as described above. The compound has a mucin-type sugar chain binding site and an integrin recognition site, and therefore can bind to a mucin-type sugar chain and recognize integrin.

The type of binding of the polypeptide comprising an integrin recognition sequence to a mucin-type sugar chain-binding lectin is not particularly limited. For example, the binding may be an amide bond between the amino group of the lectin comprising a mucin-type sugar chain-binding site and the carboxyl group of the polypeptide comprising an integrin recognition site.

A linker may be present between the lectin and the polypeptide. For example, the lectin and the polypeptide may be bound using a known crosslinking agent. Examples of such crosslinking agents include BMPS, EMCS, GMBS, MBS, LC-SMCC, SMCC, SMPB, SMPH, Sulfo-EMCS, Sulfo-MBS, Sulfo-SMCC, Sulfo-GMBS, Sulfo-SMPB, and the like.

Beads may be interposed between the mucin-type sugar chain-binding lectin and the polypeptide comprising an integrin recognition sequence. The beads for use are not particularly limited, and examples of usable beads include Sepharose beads. For example, when Sepharose beads containing NHS groups are used to form a bond via Sepharose beads, the NHS groups of Sepharose beads and NH₂ groups of the lectin and the peptide are reacted to form amide bonds, whereby the polypeptide comprising an integrin recognition sequence and the mucin-type sugar chain-binding lectin can be bound via the beads. Commercially available products can be used as Sepharose beads containing NHS groups. For example, NHS-activated beads (GE Healthcare) can be used.

The polypeptide comprising an integrin recognition sequence and the mucin-type sugar chain-binding lectin are bound at a site where the mucin-type sugar chain binding site and the integrin recognition site are not impaired. More specifically, the lectin and the polypeptide are bound in such a manner that mucin-type sugar chain-binding ability of the mucin-type sugar chain-binding lectin and integrin recognition ability of the polypeptide comprising an integrin recognition sequence are not impaired.

The binding ratio of the polypeptide comprising an integrin recognition sequence to the mucin-type sugar chain-binding lectin can be suitably selected from the range in which the effect of the present invention is provided. The molar ratio of the lectin to the polypeptide is preferably about 1:0.01 to about 1:10. When the polypeptide consists of 100 amino acid residues (particularly about 3 to about 20 amino acid residues), the molar ratio of the lectin to the polypeptide is more preferably about 1:0.1 to about 1:10, still more preferably about 1:0.3 to about 1:10, and still further more preferably about 1:3 to about 1:10. When the polypeptide is a protein such as fibronectin, vitronectin, or laminin (a polypeptide consisting of 500 amino acid residues or more, particularly 2,000 amino acid residues or more), the molar ratio of the lectin to the polypeptide is preferably about 1:0.01 to about 1:0.1, even more preferably about 1:0.02 to about 1:0.1.

The prophylactic agent for periodontal disease of the present invention contains, as an active ingredient, a compound having both of a mucin-type sugar chain-binding site and an integrin recognition site. The prophylactic agent for periodontal disease of the present invention may consist only of the compound, or may contain the compound as an active ingredient and further contain other pharmaceutically or hygienically acceptable carrier(s).

The amount of the compound in the prophylactic agent for periodontal disease is not particularly limited, insofar as the effect of the present invention is not impaired. For example, the amount of the compound is preferably 0.1 to 100 mass %, more preferably 1 to 99 mass %, and even more preferably about 50 to about 99 mass %.

The kind and amount of the carrier can be suitably selected and adjusted according to the dosage form of the prophylactic agent for periodontal disease of the present invention, insofar as the effect of the present invention is not impaired.

The form of the prophylactic agent for periodontal disease is not particularly limited. The prophylactic agent may be formed into a liquid, an emulsion, a paste, a cream, a powder, a granule, etc. Examples of the carrier include excipients such as lactose, glucose, starch, and crystalline cellulose; binders such as gelatin, carboxymethyl cellulose, and hydroxypropylcellulose; disintegrators such as sodium carboxymethylcellulose and low substituted hydroxypropylcellulose; surfactants such as polyoxyethylene sorbitan fatty acid esters; and lubricants such as stearate. Examples of diluents include commonly used solubilizers and buffers, such as water, ethyl alcohol, propylene glycol, and polyoxyethylene sorbitan fatty acid esters.

Moreover, the prophylactic agent for periodontal disease of the present invention can be used as a component of other products for use in the oral cavity, such as oral compositions, tooth-cleaning apparatuses, and chewing toys.

The compound used as an active ingredient of the prophylactic agent for periodontal disease of the present invention comprises both of a mucin-type sugar chain-binding site and an integrin recognition site. Therefore, the compound can greatly retain the integrin recognition site on buccal cells, and inhibit binding of FimA type II fimbrial protein to integrin for a long period of time. Furthermore, the compound has a high inhibitory effect on binding between P. gingivalis and gingival cells, can inhibit activation of a TNF receptor, and can effectively inhibit the development of periodontal disease and the progression of inflammation.

Since the prophylactic agent for periodontal disease of the present invention contains the above compound as an active ingredient, the prophylactic agent can inhibit binding of type II FimA fimbrial protein and integrin for a long period of time. Furthermore, the prophylactic agent has a high inhibitory effect on binding of P. gingivalis to gingival cells, and can effectively inhibit the development of periodontal disease and the progression of inflammation.

The prophylactic agent for periodontal disease of the present invention can be used for humans as well as for mammals other than humans, such as pets, livestock, and like animals (particularly dogs, cats, monkeys, cows, horses, pigs, sheep, etc.). Further, the prophylactic agent for periodontal disease of the present invention is useful not only for periodontal disease patients and periodontal disease animals, but also for other humans and animals who have not yet suffered from periodontal disease, to prevent the development of periodontal disease. For this preventive purpose, it is particularly preferable to apply the compound to humans and animals who carry, in the oral cavity, P. gingivalis carrying the type II fimA gene.

The amount of the compound comprising both of a mucin-type sugar chain-binding site and an integrin recognition site, which is contained as an active ingredient in the prophylactic agent for periodontal disease of the present invention, is not particularly limited. The compound can be used in a prophylactically effective amount suitably selected. For example, the prophylactically effective amount may be selected so that the amount of application to the oral cavity, based on the amount of the mucin-type sugar chain-binding lectin contained in the compound, is about 0.1 to about 10 μmol (preferably about 0.5 to about 5 μmol) per adult per day.

The prophylactic agent for periodontal disease can be applied to the oral cavity in a manner suitably selected according to the form of the prophylactic agent for periodontal disease. For example, the prophylactic agent in the form of a liquid can be applied by rinsing the inner mouth therewith. The prophylactic agent in the form of a paste or a gel can be directly applied to the gums, or put on a toothbrush and applied by brushing the teeth and gums with the toothbrush.

2. Oral Composition

The composition for the oral cavity of the present invention contains the prophylactic agent for periodontal disease, and is used for preventing the development of periodontal disease and preventing the worsening of a periodontal disease condition or lesion (progression prevention). When the prophylactic agent for periodontal disease is a composition, the prophylactic agent for periodontal disease per se can be the composition for the oral cavity of the present invention.

The prophylactic agent for periodontal disease may be singly incorporated into the composition, or may be mixed with a pharmaceutically acceptable carrier. Alternatively, the prophylactic agent in a form bound to a pharmaceutically acceptable carrier may be incorporated into the composition.

Examples of carriers include abrasives, nylon fibers, etc. Examples of abrasives include calcium carbonate, calcium phosphate, calcium sulfate, magnesium carbonate, magnesium hydroxide, etc. Although the shape of the carrier is not particularly limited, the carrier may be, for example, fine particles, fibrous, spherical, stringy, etc.

The type of bonding between the prophylactic agent for periodontal disease agent and the carrier is not particularly limited. For example, when an abrasive, which is a porous material, is used, a method of filling a protein or a peptide in the abrasive can be used. When nylon fibers are used, electrostatic and hydrophobic bonding can be used.

The mixing or binding ratio of the prophylactic agent for periodontal disease and the carrier can be suitably determined according to the form of the composition, and the type of peptide or protein, etc.

Mixing with or binding to the carrier is advantageous because the prophylactic agent for periodontal disease can be more easily retained for an appropriate period of time, and removed when the prophylactic agent becomes unnecessary or when excessive retention needs to be prevented.

Examples of the composition for the oral cavity include oral washes, toothpaste compositions, liquid tooth-cleaning compositions, and the like. Furthermore, the form of the composition for the oral cavity is not particularly limited. For example, the composition may be in the form of a liquid, an emulsion, a paste, a cream, or the like.

The amount of the compound contained as the active ingredient in the composition for the oral cavity is not particularly limited, insofar the effect of the present invention is provided. The amount of the compound is typically about 1 to about 10 wt %, and preferably about 1 to about 5 wt %, based on the total amount of the composition for the oral cavity. The composition for the oral cavity (particularly oral washes, liquid tooth-cleaning compositions, and like liquid compositions for the oral cavity) is preferably such that the compound as the active ingredient preferably contains a lectin in a concentration of 15 to 150 μM, and more preferably 50 to 100 μM. When a lectin is administered in a large amount, cytotoxicity may develop. However, even if a lectin in the above-mentioned concentration is swallowed by mistake, no particular problems will arise. It is, however, usually preferable that the composition for the oral cavity of the present invention be expelled from the mouth after the application.

The composition for the oral cavity may further contain other medicinally effective ingredients in addition to the prophylactic agent for periodontal disease and the carrier. Examples of other medicinally effective ingredients include minocycline hydrochloride, chlorhexidine, phenolic compounds, povidone iodine, and the like.

The composition for the oral cavity can be produced according to a known method. For example, the composition can be produced by mixing the compound as an active ingredient with a carrier suitably selected, as necessary. The amount of the carrier to be used can also be suitably selected.

Since the composition for the oral cavity of the present invention contains a prophylactic agent for periodontal disease, the composition has a long-term inhibitory effect on binding of type II FimA fimbrial protein of a periodontal pathogen to integrin, and is excellent in preventing and inhibiting the development and progression of periodontal disease, whereby the composition is also excellent in long-term plaque control, control of bad breath caused by the periodontal disease-causing bacteria, etc.

3. Tooth-Cleaning Apparatus

The tooth-cleaning apparatus according to the present invention is an instrument for cleaning teeth. Examples of the tooth-cleaning apparatus include toothbrushes, dental flosses, interdental brushes, interdental stimulators, rubber tips, oral cavity cleaners, etc. The “oral cavity cleaner” is an apparatus for removing plaque and food debris from the oral cavity (particularly between the teeth or in periodontal pockets) by jetting water.

The tooth-cleaning apparatus of the present invention comprises the above-mentioned prophylactic agent for periodontal disease, and is used for preventing the development of periodontal disease or preventing the worsening of a periodontal disease condition or lesion.

The manner of incorporating the prophylactic agent for periodontal disease into the tooth-cleaning apparatus of the present invention is not particularly limited, insofar as the effect of the present invention is provided. For example, a toothbrush may be designed to contain the prophylactic agent for periodontal disease therein, and release the prophylactic agent in the oral cavity when brushing teeth. More specifically, for example, a toothbrush may be designed to comprise: a toothbrush handle in which the prophylactic agent for periodontal disease is contained in a container; and a passage communicating between the container and a brush section, so that the prophylactic agent for periodontal disease is gradually pushed out from the container and transported through the passage to the brush section by means of a motor or a piston. Another example may be a tooth-cleaning apparatus made of a known material, and partially or entirely coated with the prophylactic agent for periodontal disease by application or adhesion of the prophylactic agent onto the surface thereof. Specific examples thereof include a tooth-cleaning apparatus comprising a toothbrush whose brush portion is coated with the prophylactic agent for periodontal disease.

The tooth-cleaning apparatus may contain the prophylactic agent for periodontal disease that is in a form mixed or bound to a suitable carrier. Examples of usable carriers may be the same as mentioned above in the section of the Oral Composition.

The amount of the prophylactic agent for periodontal disease of the invention in the tooth-cleaning apparatus can also be suitably selected from the range in which the effect of the present invention can be provided.

Since the tooth-cleaning apparatus of the present invention contains a prophylactic agent for periodontal disease of the present invention, the apparatus has a long-term inhibitory effect on binding of type II FimA fimbrial protein of periodontal disease-causing bacteria to integrin, and is excellent in preventing or inhibiting the development and progression of periodontal disease, whereby the apparatus is also excellent in long-term plaque control, control of bad breath caused by the periodontal pathogen, etc.

4. Method for Inhibiting Binding Between P. Gingivalis and Gingival Cells

The present invention is also directed to a method of inhibiting binding of the periodontal disease-causing bacteria, P. gingivalis, to gingival cells by applying the prophylactic agent for periodontal disease comprising, as an active ingredient, the compound comprising both of a mucin-type sugar chain binding site and an integrin recognition site. As described above, this method can inhibit binding of P. gingivalis to gingival cells for a relatively long period of time. Inhibition of binding between P. gingivalis and gingival cells can prevent the development and progression of periodontal disease. The method can also suppress the production of cytokine, and inhibit TNF receptor activation to thereby suppress inflammation.

The active ingredient of the prophylactic agent for periodontal disease used in this method, preparation of the prophylactic agent for periodontal disease, the subject, the amount of application, the application method, etc. may be as described above.

### ADVANTAGEOUS EFFECTS OF INVENTION

The prophylactic agent for periodontal disease of the present invention comprises, as an active ingredient, a compound having a mucin-type sugar chain-binding site and an integrin recognition site. Due to the function of the mucin-type sugar chain-binding site, the compound has high retentivity on the acquired pellicle. As a result, the integrin recognition site can also retain on the acquired pellicle for a prolonged period of time. Therefore, the compound can inhibit binding of type II FimA fimbrial protein to integrin for a long period of time. Thus, the prophylactic agent for periodontal disease of the present invention can potently inhibit binding of P. gingivalis to gingival cells. Further, the prophylactic agent can suppress production of cytokine and inhibit activation of TNF receptors, and thus has a highly effective inhibitory effect on the development of periodontal disease and the progression of inflammation.

Furthermore, the composition for the oral cavity and the tooth-cleaning apparatus of the present invention contain a prophylactic agent for periodontal disease of the present invention. Therefore, the composition and the tooth-cleaning apparatus have a long-term inhibitory effect on binding of type II FimA fimbrial protein of periodontal pathogen to integrin, and can effectively prevent and inhibit the development and progression of periodontal disease.

### BRIEF DESCRIPTION OF DRAWINGS

FIG. 1 shows the results of an investigation of the binding of FimA to cell surfaces expressing integrin α5β1. In FIG. 1, the white portions indicate the background, whereas the black portions indicate bound cells. The expression of integrin α5β1 on the cells is confirmed in the graphs in the upper row. The binding of type II FimA fimbrial protein to cells is confirmed in the graphs in the lower row.

FIG. 2 shows the results of using an experimental system containing immobilized cells to which FimA was bound, to investigate the binding inhibitory effect of no treatment (untreated), and the binding inhibitory effect when (i) SBA beads (SBA alone), (ii) fibronectin (fibronectin alone), (iii) fibronectin−SBA beads (fibronectin+SBA), (iv) RGD peptide (RGD peptide alone), or (v) RGD peptide−SBA beads (RGD+SBA) were allowed to coexisted with FimA.

FIG. 3 shows the results of using an experimental system containing immobilized FimA to which α5β1 integrin-expressing cells were bound, to investigate the binding inhibitory effect of no treatment (untreated), and the binding inhibitory effect when (i) SBA beads (SBA alone), (ii) fibronectin (fibronectin alone), (iii) fibronectin−SBA beads (fibronectin+SBA), (iv) RGD peptide (RGD peptide alone), or (v) RGD peptide−SBA beads (RGD peptide+SBA) were allowed to coexisted with FimA.

FIG. 4 shows the results of using an experimental system containing immobilized cells to which P. gingivalis carrying type II fimA fimbrial protein was bound, to investigate the binding inhibitory effect of no treatment (untreated), and the binding inhibitory effect when (i) SBA beads (SBA alone), (ii) fibronectin SBA beads (fibronectin−SBA), or (iii) RGD peptide−SBA beads (RGD−SBA) were allowed to coexist with P. gingivalis.

FIG. 5 shows the results of using an experimental system containing immobilized FimA to which α5β1 integrin-expressing cells were bound, to investigate the binding inhibitory effect of no treatment (untreated), and the binding inhibitory effect when (i) SBA beads (SBA beads), (ii) RGD peptide (RGD peptide), (iii) RGD peptide−SBA beads (SBA+RGD peptide), (iv) WGA beads (WGA), or (v) RGD peptide-WGA beads (WGA+RGD peptide) were allowed to coexist with α5β1 integrin-expressing cells.

FIG. 6 shows the results of using an experimental system containing immobilized FimA to which α5β1 integrin expressing cells were bound, to investigate the influence of (1) no treatment and the influence of (i) SBA beads (SBA alone), (ii) RGD peptide (RGD peptide), (iii) RGD peptide−SBA beads (SBA−RGD peptide), (iv) RGD peptide−SBA beads and glucose (SBA−RGD+glucose), (v) RGD peptide−SBA beads and N-acetylgalactosamine (SBA−RGD+GalNAc), or (vi) N-acetylgalactosamine alone (GalNAc alone) coexisting with α5β1 integrin on the SBA binding inhibitory effect of monosaccharide.

FIG. 7 shows the results of using an experimental system containing immobilized cells that were not treated (untreated), or to which (2) lipopolysaccharide (LPS), (3) TNF alpha, (4) P. gingivalis carrying type II FimA fimbrial protein, or (5) FimA was added, to investigate the TGF-β cytokine production inhibitory effect of (i) no treatment (untreated), and the TGF-β cytokine production inhibitory effect when (ii) SBA beads (SBA alone), (iii) fibronectin−SBA beads (fibronectin+SBA), or (iv) RGD peptide−SBA beads (peptide+SBA) were allowed to coexist with the cells.

FIG. 8 shows the results of using an experimental system with (1) no treatment (untreated), or in which (2) SBA beads (SBA alone), (3) fibronectin SBA beads (fibronectin+SBA), or (4) RGD peptide−SBA beads (RGD peptide+SBA) were present, to investigate the TNF receptor 1 (TNFRI) phosphorylation inhibitory effect by western blotting using (i) an anti-TNFRI antibody, (ii) an anti-tyrosine phosphorylation antibody, or (iii) an anti-actin antibody.

### EXAMPLES

The present invention is described below in more detail with reference to Examples and Comparative Examples. However, the scope of the invention is not limited to these Examples.

1. Production of Soybean Lectin Beads (SBA Beads)

NHS-activated beads (Sepharose beads, manufactured by GE Healthcare Company) were added to soybean lectin (SBA; a product of Seikagaku Corporation) in an amount of 1 ml per 5 mg of the soybean lectin, and mixed. The mixture was allowed to stand at room temperature for 1 hour to bind the amino group of soybean lectin to the beads.

The binding ratio of soybean lectin to Sepharose beads was measured. The results showed that 5 mg of soybean lectin was almost completely bound to 1 ml of Sepharose beads.

The obtained conjugate of soybean lectin and beads is hereinafter referred to as “SBA beads”.

2. Production of RGD Peptide−SBA Beads or Fibronectin−SBA Beads

An RGD-peptide consisting of the amino acid sequence set forth in SEQ ID NO: 3 of the Sequence Listing (a product of Sigma Aldrich Japan, Inc.), or fibronectin (a product of Wako Pure Chemical Industries, Ltd.) was mixed with soybean lectin beads obtained in the above production process 1. Each mixture was allowed to stand at room temperature for 1 hour to bind the RGD-peptide or fibronectin to the soybean lectin beads via the carboxyl terminal of the RGD peptide or fibronectin by using EDC (1-ethyl-3-[3-dimethylaminopropyl]carbodiimide hydrochloride, manufactured by Pierce). Unbound protein or peptide was collected. Beads in which SBA beads and RGD-peptides were bound, and beads in which SBA beads and fibronectin were bound were obtained.

Hereinafter, the beads in which SBA beads and RGD-peptide are bound are referred to as “RGD peptide−SBA beads”, and the beads in which SBA beads and fibronectin are bound are referred to as “fibronectin−SBA beads”.

3. Test Cells

CHO cells on which α5β1 integrin was forcedly expressed by introducing a gene encoding α5β1 integrin (hereinafter sometimes referred to as “CHO-VLA5”), and gingival cells were used in the Binding Inhibitory Experiments described below. As the gingival cells, three types of cells, GE1, Ca9-22 and Sa3, were used. As a control, CHO cells into which α5β1 integrin was not introduced were used. GE1, Ca9-22, and Sa3 were obtained from ATCC (American Type Culture Collection).

Approximately 10,000 cells each were prepared. The expression of α5β integrin on the surface of the cells was confirmed with a flow cytometer (FACSCalibur: manufactured by Becton, Dickinson and Company) by using anti-integrin β1 antibody (clone name: 4B7R) (available from GeneTex Inc.) and FITC anti-mouse IgG antibody (available from Jackson Laboratory). The binding of FimA II-GST to each type of the cells was also confirmed with a flow cytometer by using FITC-anti-GST antibody (available from AnaSpec, Inc.). FimA II-GST is a fusion protein comprising a GST protein fused to the N-terminus of FimA II.

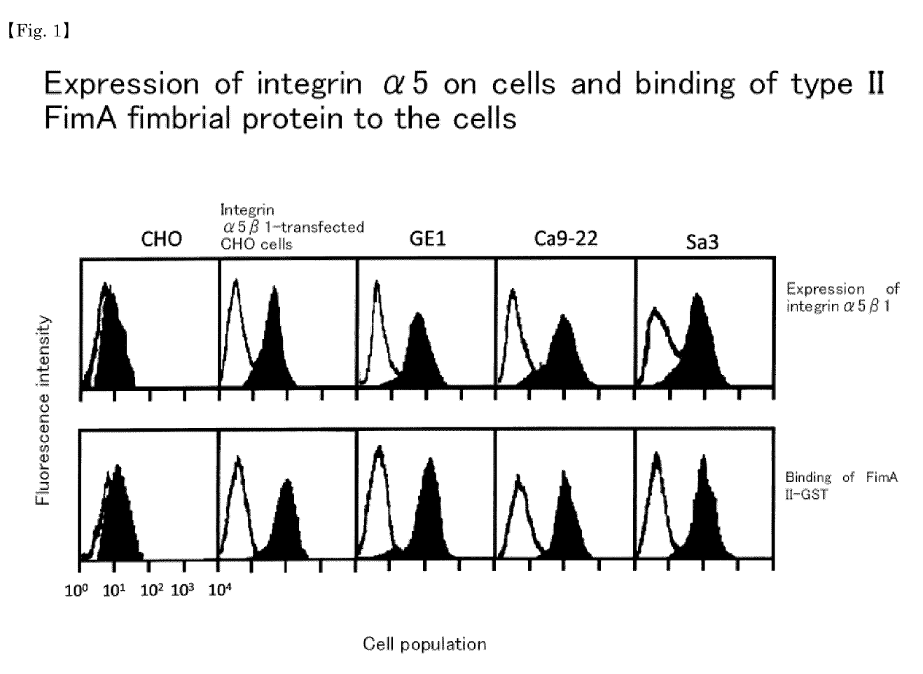

FIG. 1 shows the results.

As shown in FIG. 1, the expression of α5β1 integrin was confirmed in all types of the cells: α5β1 integrin-infected CHO cells, GE1, Ca9-22 and Sa3.

These 4 types of cells, and α5β1 integrin-uninfected CHO cells as a negative control were used as test cells in the Experiments shown below.

4. Binding Inhibitory Experiment 1

Test cells were seeded into a 96-well plate in an amount of 5×10⁵ cells. Subsequently, 50 μl of FimA II-His (10 μg/ml) was allowed to bind to the cells, and 10 μl of SBA beads, RGD peptide−SBA beads, or fibronectin−SBA beads were allowed to coexist. The plate was shaken at room temperature for 10 minutes. For comparison, an experiment in which an RGD peptide or fibronectin was allowed to coexist was performed in a similar manner. The FimA II-His is a fusion protein comprising a His tag (His 10 residues) fused to the C-terminus of FimA II protein. Strain OMZ314 (NCBI accession No. AB261607.1) was used as FimA II. The His-tagged fusion protein was produced by using the vector pET52b.

After the 96-well plate was gently washed, FimA II-His bound to the cells was detected by ELISA using an anti-His tag antibody (Cat. No. 71554-3; available from Novagen).

The fibronectin−SBA bead solution contained about 30 μM of fibronectin and about 100 μM of SBA, whereas the solution containing only fibronectin contained about 30 μM of fibronectin. These values were calculated by measuring the amount of each protein not bound to the beads during the production.

The RGD peptide−SBA bead solution contained about 30 μM of RGD peptide and about 100 μM of SBA, whereas the solution containing only an RGD peptide contained about 30 μM of RGD peptide. The SBA bead solution contained about 100 μM of SBA.

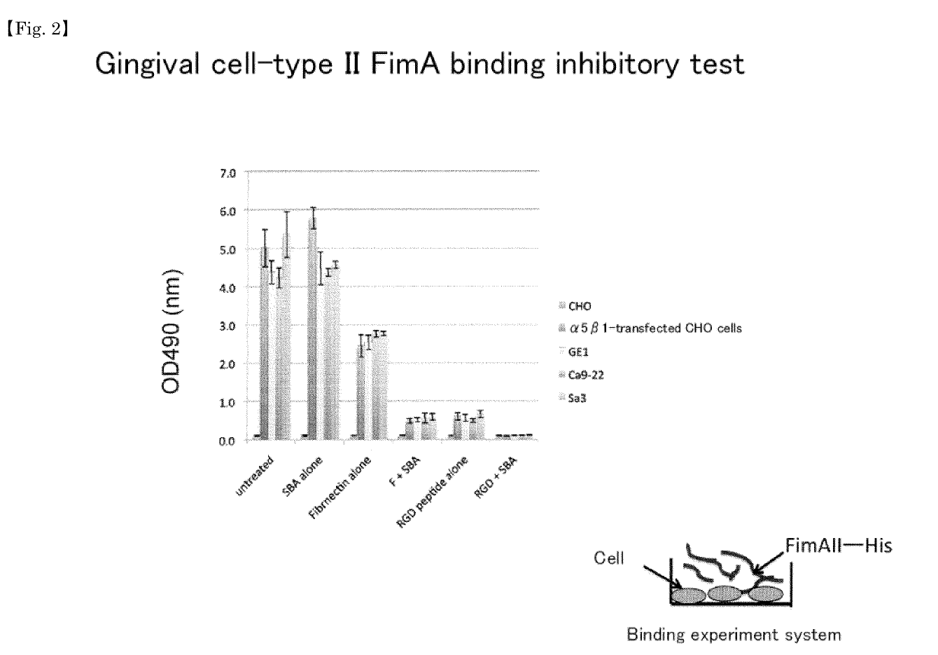

FIG. 2 shows the results.

The results of FIG. 2 show that compared to the single use of fibronectin, fibronectin−SBA beads significantly inhibited binding of FimA type II fimbrial protein to α5β1 integrin expression cells; and that compared to the single use of RGD peptide, RGD peptide−SBA beads significantly inhibited binding of FimA type II fimbrial protein to α5β1 integrin expression cells.

5. Binding Inhibitory Experiment 2

FimA II-His (10 μg/(ml)) was immobilized onto a 96-well plate, and test cells that had been fluorescently labeled with CellTracker Green CMFDA (available from Invitrogen) were added in an amount of 1×10⁶ cells and allowed to bind.

50 μl of SBA beads, RGD peptide−SBA beads, or fibronectin−SBA beads were allowed to coexist in each well, and the plate was shaken. For comparison, an experiment in which an RGD peptide or fibronectin was allowed to coexist was performed in a similar manner.

Each well of the 96-well plates was filled with serum-free Dulbecco's modified Eagle's medium (hereinafter referred to as DMEM) or with PBS, and covered with a Parafilm to prevent air from entering. The plate was turned upside-down and allowed to stand for 5 minutes. After removing the Parafilm, a supernatant containing unbound cells was removed, and bound cells were detected using a fluorescent plate reader.

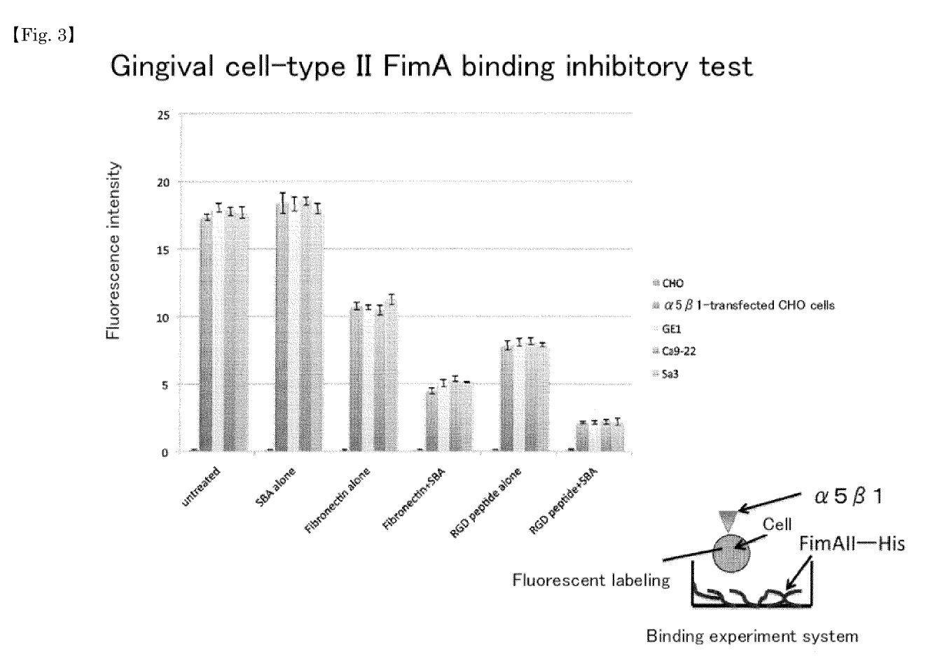

FIG. 3 shows the results.

The results of FIG. 3 show that compared to the presence of fibronectin alone, fibronectin−SBA beads significantly inhibited binding of type II FimA protein to α5β1 integrin expression cells; and that compared to the presence of RGD peptide alone, RGD peptide−SBA beads significantly inhibited binding of FimA II protein to α5β1 integrin expression cells.

The results further show that the inhibitory effect of RGD peptide−SBA beads is higher than that of fibronectin−SBA beads. This is probably because the RGD sequence site of the RGD peptide−SBA beads is more effectively exposed on the surface of SBA beads.

The results of Binding Inhibitory Experiments 1 and 2 confirmed that even when cells or FimA II is immobilized, these beads can effectively inhibit binding of FimA II protein to α5β1 integrin.

Another experiment was performed using α5β1 integrin expressing cells in a manner similar to the FimA II-His tag protein binding inhibitory experiment described above, to investigate the binding ratio of RGD peptide or fibronectin to SBA beads. The results show that among the RGD peptide−SBA beads, RGD peptide−SBA beads in which RGD peptide was bound to SBA beads in an amount of 10 to 30 μg per 100 μg of SBA beads inhibited binding most effectively; and that among the fibronectin−SBA beads, fibronectin−SBA beads in which fibronectin was bound to SBA beads in an amount of 100 to 30 μg per 100 μg of SBA beads inhibited binding most effectively.

As described above, SBA beads have a structure such that SBA is bound to beads in an amount of 5 mg per mL of the beads. Therefore, 100 μL of SBA beads contain about 500 μg of SBA.

Molecular weight of SBA: about 120,000 (a trimer)
Molecular weight of RGD peptide: about 1,000
Molecular weight of fibronectin: about 250,000
From these values, the binding molar ratio of SBA to RGB peptide in the “RGD peptide−SBA beads in which RGD peptide is bound to SBA beads in an amount of 10 to 30 μg per 100 μL of the SBA beads” can be calculated to be in the range of 1:2.4 to 1:7.2. Further, the binding molar ratio of SBA to fibronectin in the “fibronectin−SBA beads in which fibronectin is bound to SBA beads in an amount of 100 to 30 μg per 100 μL of the SBA beads” can be calculated to be in the range of 1:0.096 to 1:0.0288.

6. Binding Inhibitory Experiment 3

Test cells were seeded into a 96-well plate in an amount of 5×10⁵ cells. The next day, 10 μl of P. gingivalis carrying FimA type II fimbrial protein was allowed to bind, and 30 μl each of SBA beads, RGD peptide−SBA beads, and fibronectin−SBA beads were added. The plate was shaken at room temperature for 10 minutes.

The 96-well plate was gently washed. Fluorescently labeled P. gingivalis carrying type II FimA fimbrial protein bound to the cells was detected using a fluorescence detector. P. gingivalis was fluorescently labeled by using an FITC Labeling Kit (manufactured by Kirkegaard & Perry Laboratories, Inc.) after P. gingivalis was immobilized with formalin.

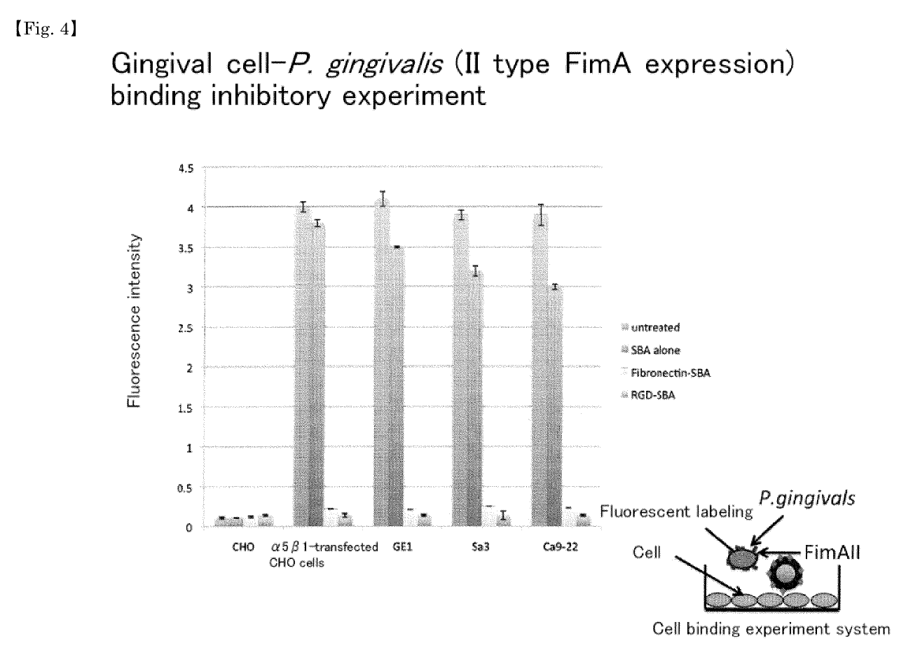

FIG. 4 shows the results.

The results of FIG. 4 show that RGD peptide−SBA beads and fibronectin−SBA beads inhibited binding of P. gingivalis carrying type II FimA fimbrial protein to gingival cells.

Compared to SBA beads, RGD peptide−SBA beads and fibronectin−SBA beads exhibited a remarkably high binding inhibitory effect. However, SBA beads also slightly inhibited binding. These results suggest that a mucin-type sugar chain-binding lectin as well as RGD peptide or fibronectin has an inhibitory effect on the binding of P. gingivalis carrying type II FimA fimbrial protein and gingival cells.

7. Comparison with Other Lectins

An investigation was conducted using a wheat germ lectin (wheat germ agglutinin; hereinafter sometimes referred to as “WGA”) in addition to SBA.

FimA II-His (10 μg/ml) was immobilized onto a 96-well plate, and test cells that had been fluorescently labeled with CellTracker Green CMFDA (manufactured by Invitrogen) were bound in an amount of 1×10⁶ cells.

Subsequently, 10 μL of SBA beads, RGD peptides, RGD peptide−SBA beads, WGA beads, or RGD peptide-WGA beads were allowed to coexist, and the plate was shaken.

The WGA beads are beads prepared in the same manner as in the production of SBA beads described above in Item 1, except for using WGA in place of SBA. The RGD peptide-WGA beads are beads prepared in the same manner as in the production of RGD peptide−SBA beads described above in Item 2, except for using WGA in place of SBA.

The 96-well plate was filled with PBS. After unbound cells were removed, bound cells were detected using a fluorescent plate reader.

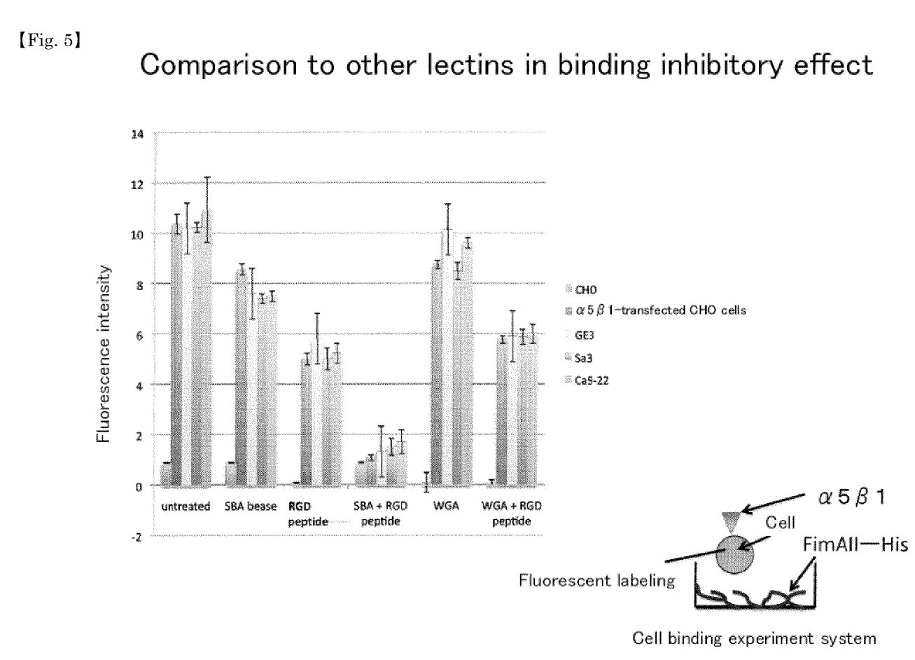

FIG. 5 shows the results.

WGA has the property of binding to chitooligosaccharide carrying D-GlcNAc and a complex-type asparagine-linked sugar chain having an N-acetyllactosamine structure (N-type sugar chain). The results of FIG. 5 show that compared to WGA, SBA more potently inhibits the interaction between gingival cells and FimA II. Thus, the results suggest that the use of a mucin-type sugar chain having a GalNAcα1-3Gal structure enhances the retention of RGD or fibronectin.

8. Effect of Monosaccharide on Binding Inhibitory Effect

FimA II-His (10 μg/ml) was immobilized onto a 96-well plate, and test cells that had been fluorescently labeled with CellTracker Green CMFDA (Invitrogen) were bound thereto in an amount of 1×10⁶ cells.

SBA beads, RGD peptide, or RGD peptide−SBA beads were allowed to coexist in the wells in an amount of 10 μl, and the plate was shaken. In addition to the RGD peptide−SBA beads, glucose or N-acetylgalactosamine (GalNAc) was allowed to coexist therewith in a final concentration of 50 μg/ml. For comparison, wells of a 96-well plate in which no beads were present and only N-acetylgalactosamine was present in a final concentration of 50 μg/ml were also prepared.

The 96-well plate was filled with PBS. After unbound cells were removed, bound cells were detected using a fluorescent plate reader.

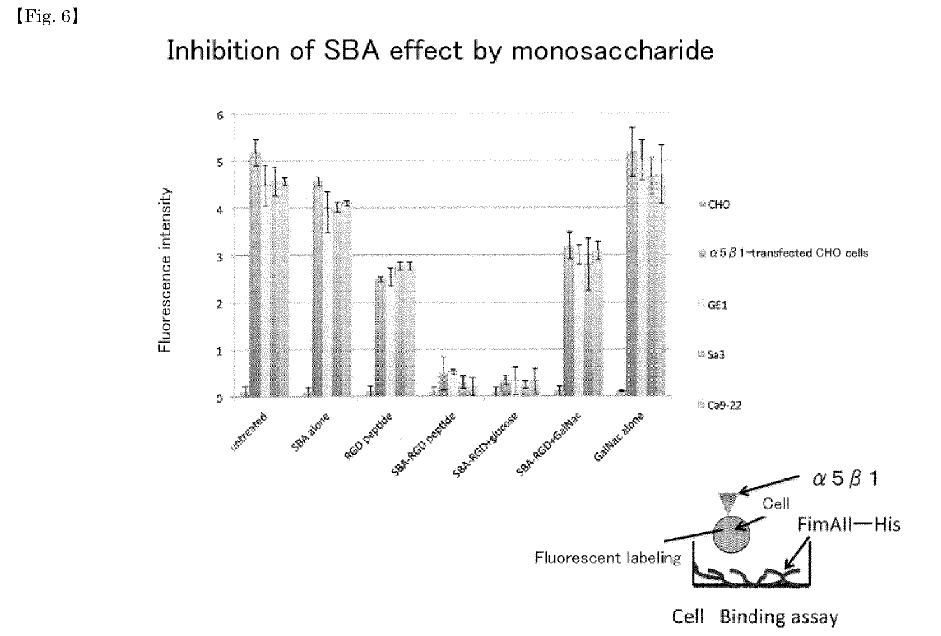

FIG. 6 shows the results.

The results of FIG. 6 show that the coexistence of glucose did not inhibit the RGD peptide−SBA binding inhibitory effect, whereas the coexistence of N-acetylgalactosamine did so. SBA is known to bind to an N-acetylgalactosamine structure. Thus, the results indicate that SBA bound to the N-acetylgalactosamine structure contributes to inhibition of binding between gingival cells and FimA II.

9. Confirmation of Cytokine Production

Ca9-22 cells were seeded into a 96-well plate in an amount of 5×10⁵ cells. The next day, (1) a lipopolysaccharide (hereinafter also referred to as “LPS”; 10 ng/ml), (2) TNF-α(10 ng/ml), (3) P. gingivalis carrying FimA type II fimbrial protein (5 μl), or (4) FimA II-GST (10 μg/ml) was added as a reagent. The parenthesized numerals in the above sentence indicate the final concentrations in the media containing the reagents to 50 μl of the medium.

SBA beads, RGD peptide−SBA beads, or fibronectin−SBA beads were allowed to coexist therewith in an amount of 30 μl, and the plate was rocked. The amount of TGF-β produced was then measured.

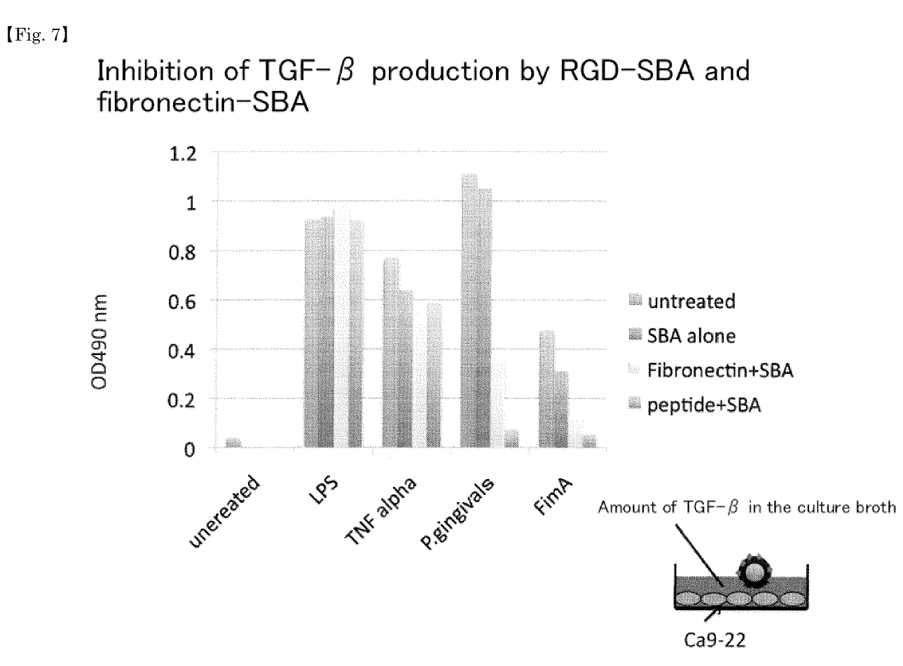

FIG. 7 shows the results.

The results show that when SBA beads, RGD peptide−SBA beads, or fibronectin−SBA beads were allowed to coexist in the plate treated with (1) LPS or (2) TNF-α, production of TGF-β was observed as in the system in which no beads were present. These results indicate that RGD peptide−SBA beads and fibronectin−SBA beads hardly affect the inflammatory reaction of (1) LPS or (2) TNF-α.

In contrast, in the plate containing (3) P. gingivalis, when no beads were present or SBA beads were allowed to coexist, production of TGF-β was detected, whereas when RGD peptide−SBA beads or fibronectin SBA beads were allowed to coexist, production of TGF-β was inhibited. Similarly, in the plate containing (4) FimA II, when no beads were present or SBA beads were allowed to coexist, production of TGF-β was detected; whereas when RGD peptide−SBA beads or fibronectin−SBA beads were allowed to coexist, production of TGF-β was inhibited.

These results confirmed that RGD peptide−SBA beads and fibronectin−SBA beads inhibited the production of TGF-β.

10. Effect of TNFRI (TNF-α Receptor Type I) on Phosphorylation

Test cells were seeded into a 96-well plate in an amount of 5×10⁵ cells. The next day, each well was washed with DMEM, and then P. gingivalis carrying type II FimA fimbrial protein (10 μl) was added as a reagent. The numerical value in the parentheses indicates the final concentration in the medium containing the reagent.

SBA beads, RGD peptide−SBA beads, or fibronectin−SBA beads were allowed to coexist therewith in an amount of 30 μl, and the plate was shaken. After each plate was washed well with PBS, cells were dissolved in 1% NP-40, and subjected to western blotting using an anti-TNFRI antibody, anti-tyrosine phosphorylation antibody, and anti-actin antibody.

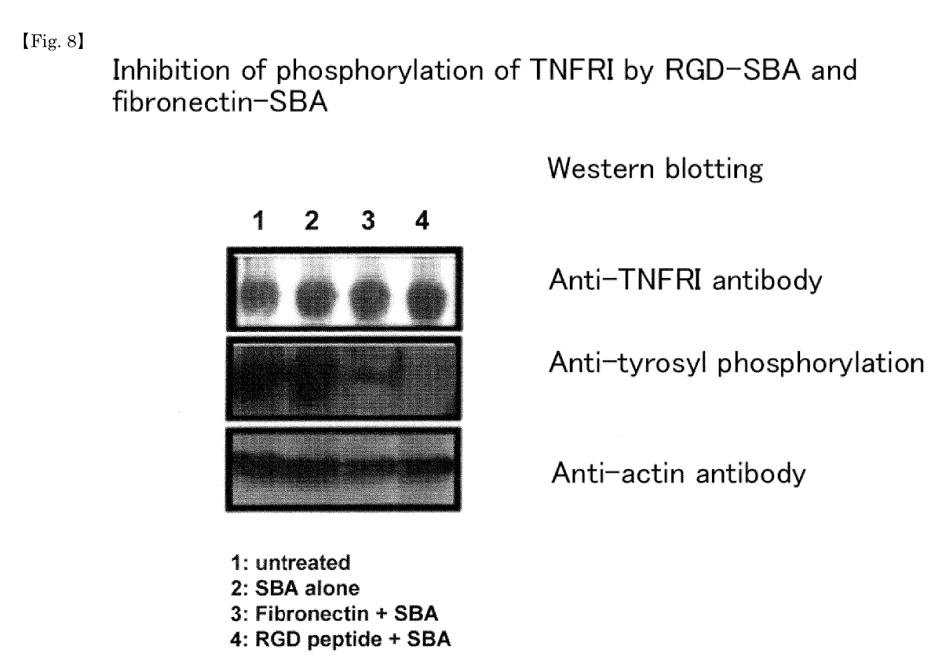

FIG. 8 shows the results.

The results show that when fibronectin−SBA beads or RGD peptide−SBA beads were added, phosphorylated tyrosine of the activation receptor was not detected. The results revealed that RGD peptide−SBA beads and fibronectin−SBA beads inhibit binding of type II FimA fimbrial protein to α5β1 integrin on gingival cells to thereby inhibit the activation of the inflammatory cytokine TNF receptor. The results thus suggested that RGD peptide−SBA beads and fibronectin−SBA beads might inhibit inflammation caused by FimA of gingival cells.

[Sequence Listing]

1. A compound comprising a mucin-type sugar chain-binding lectin and a polypeptide bound thereto, the polypeptide comprising an integrin recognition sequence.

2. The compound according to claim 1, wherein the mucin-type sugar chain-binding lectin is at least one lectin selected from the group consisting of mushroom lectin, Dolichos biflorus lectin, peanut lectin, and soybean lectin.

3. The compound according to claim 1, wherein the polypeptide comprising the integrin recognition sequence comprises an amino acid sequence set forth in SEQ ID NO: 1 or 2.

4. The compound according to claim 3, wherein the polypeptide comprising the amino acid sequence set forth in SEQ ID NO: 1 is at least one member selected from the group consisting of the polypeptide consisting of the amino acid sequence set forth in SEQ ID NO: 3, fibronectin, vitronectin, and laminin.

5. The compound according to claim 1 comprising, as an active ingredient, a compound in which a mucin-type sugar chain-binding lectin and a polypeptide comprising an integrin recognition sequence are bound via beads.

6. The compound according to claim 1 comprising, as an active ingredient, a compound in which a mucin-type sugar chain-binding lectin and a polypeptide comprising an integrin recognition sequence are bound at a lectin:polypeptide molar ratio of 1:0.01 to 1:10.

7. A composition for the oral cavity comprising the compound of claim 1.

8. A tooth-cleaning apparatus comprising the composition for the oral cavity of claim 7.

9. A method for inhibiting binding of Porphyromonas gingivalis to gingival cells, comprising applying to the oral cavity of a human or animal subject a pharmaceutically effective amount of a compound comprising a mucin-type sugar chain-binding lectin and a polypeptide bound thereto, the polypeptide comprising an integrin recognition sequence.

10. The method according to claim 9, wherein the mucin-type sugar chain-binding lectin is at least one type of lectin selected from the group consisting of mushroom lectin, Dolichos biflorus lectin, peanut lectin, and soybean lectin.

11. The method according to claim 9, wherein the polypeptide comprising the integrin recognition sequence comprises an amino acid sequence set forth in SEQ ID NO: 1 or 2.

12. The method according to claim 11, wherein the polypeptide comprising the amino acid sequence set forth in SEQ ID NO: 1 is at least one member selected from the group consisting of the polypeptide consisting of the amino acid sequence set forth in SEQ ID NO: 3, fibronectin, vitronectin, and laminin.

13. The method according to claim 9, wherein the compound comprising the mucin-type sugar chain-binding lectin and the polypeptide comprising an integrin recognition sequence is a compound in which the lectin and the polypeptide are bound via beads.

14. The method according to claim 9, wherein the compound comprising the mucin-type sugar chain-binding lectin and the polypeptide comprising an integrin recognition sequence is a compound in which the lectin and the polypeptide are bound at a lectin:polypeptide molar ratio of 1:0.01 to 1:10.

15. The method according to claim 9, wherein the human or animal subject is a periodontal disease patient or a periodontal disease mammal.

16. The method according to claim 9, wherein the effective amount is such that the mucin-type sugar chain-binding lectin contained in the compound is administered in an amount of 0.1 to 10 μmol per day.


SEQUENCE LISTING (3 sequences)

SEQ ID NO: 1 - protein, 3 aa, Homo sapiens
RGD

SEQ ID NO: 2 - protein, 5 aa, Homo sapiens
RHSRN

SEQ ID NO: 3 - protein, 10 aa, Artificial
  synthetic peptide
RGDSPASSKP